(19) United States
(12) Patent Application Publication
(10) Pub. No.: US 2023/0237652 A1
(43) Pub. Date: Jul. 27, 2023

(54) VENOUS COMPRESSION SITE IDENTIFICATION AND STENT DEPLOYMENT GUIDANCE, AND ASSOCIATED DEVICES, SYSTEMS, AND METHODS

(71) Applicant: KONINKLIJKE PHILIPS N.V., EINDHOVEN (NL)

(72) Inventors: Molly Lara FLEXMAN, MELROSE, MA (US); Grzegorz Andrzej TOPOREK, CAMBRIDGE, MA (US); Ashish Sattyavrat PANSE, BURLINGTON, MA (US); Jochen KRUECKER, ANDOVER, MA (US); Jake MERTENS, MAPLE GROVE, MN (US); Andrew John PELTOMA, SAINT MICHAEL, MN (US)

(21) Appl. No.: 18/023,829

(22) PCT Filed: Aug. 26, 2021
(86) PCT No.: PCT/EP2021/073572

§ 371 Date: Feb. 28, 2023

Related U.S. Application Data
(60) Provisional application No. 63/072,982, filed on Sep. 1, 2020.

(51) Int. Cl. G06T 7/00; G16H 30/40; G06T 7/73

(52) U.S. Cl. CPC G06T 7/0012; G16H 30/40; G06T 7/73; G06T 2207/20084; G06T 2207/20081; G06T 2207/10132; G06T 2207/10116; G06T 2207/20021; G06T 2207/30104

(57) ABSTRACT

A system includes a processor circuit configured for communication with an external imaging device. The processor circuit receives, from the external imaging device, an image comprising a blood vessel within a patient. The processor circuit determines, using the image, a first location of the blood vessel with a restriction in blood flow caused by compression of the blood vessel by an anatomical structure within the patient and different than the blood vessel. The processor circuit generates a first graphical representation associated with the restriction. The processor circuit outputs, to a display in communication with the processor circuit, a screen display. The screen display includes the image and the first graphical representation at the first location of the blood vessel in the image.

### TECHNICAL FIELD

The present disclosure relates generally to identifying and treating blood flow occlusions within a patient. In particular, a deep learning network may be trained to identify regions of venous compression within venogram and intravascular ultrasound (IVUS) images and recommend types and placements of stents within a constricted vessel.

### BACKGROUND

Compressive venous disease (e.g. May-Thurner) occurs when bones, ligaments, or arteries compress the iliac vein and inhibit venous return. There are multiple venous compression syndromes, including Paget-Schroetter syndrome, Nutcracker syndrome, May-Thurner syndrome, and popliteal venous compression, amongst others. Unlike other vascular diseases, these syndromes are usually seen in young, otherwise healthy individuals and can lead to significant overall morbidity. Because lesions can be highly fibrotic, angioplasty alone is not an effective therapy. A majority of patients with iliofermoral deep vein thrombosis have proximal venous stenosis, which is most effectively treated with stenting.

Stenting involves placing an expandable, cylindrical device within a constricted vessel to reopen the vessel and regain blood flow. Selection and positioning of the optimal stent can be complex. Almost all stents exhibit a tradeoff of flexibility and strength. Inflexible stents must be placed with care across tortuous segments. Given the variability in anatomical distribution and extent of disease, one venous stent design may not be best suited for all conditions. In addition, not all stents should be positioned in the same location or with the same method, depending on the properties of the stent and the patient anatomy. Some stents have certain regions of optimal strength, foreshortening during deployment, and a limited selection of diameters and lengths. Anatomical structures, such as the inguinal ligament, adjacent to arteries can influence the optimal positioning of the stent to have maximum strength.

In addition to the complexities of properly selecting and positioning a stent, in regions at or around the iliac vein, certain anatomical features affecting venous compression are visible only with different imaging techniques. For example, the inguinal ligament, a common cause of peripheral stenosis, is not seen in x-ray.

### SUMMARY

Embodiments of the present disclosure are systems, devices, and methods for identifying venous compression sites in a patient's anatomy, as well as recommending to a physician a type of stent to place and the location to place the recommended stent. This advantageously provides guidance to the physician about where a blood flow is blocked in vessel, as well as how to treat the blockage so that blood flow is restored. A system configured to perform these steps may include an x-ray imaging device and an intravascular ultrasound (IVUS) imaging device, both in communication with a control system. The control system may include a processor configured to train and implement a deep learning network. The deep learning network receives as inputs an x-ray venogram image from the x-ray device, one or more IVUS images from the IVUS imaging device, and any other patient information including patient history. The deep learning network may then output multiple regions or classes, such as the location of various anatomical features within a patient's anatomy, such as the location of the iliac artery crossing over the iliac vein, locations of stenosis, and/or anatomical landmarks that can be used to determine the location of an inguinal ligament (e.g., where the ligament compresses the iliac vein). These outputs may be overlaid over the input venogram image and displayed to a user. The deep learning network may be a convolutional neural network.

The outputs of the deep learning network may be combined with additional metrics from the IVUS imaging device and/or the x-ray imaging device to recommend a type of stent to a physician using, e.g., a lookup table that reflects expert guidance about the selection of a particular stent and the placement of the particular stent at the occlusion site. For example, the locations of venous compression, along with the vessel diameter of the iliac vein or other metrics, may be used to identify a recommended stent. Based on characteristics of the recommended stent, such as diameter, length, flexibility, foreshortening, and regions of maximum strength, as well as the previously mentioned features of the patient's anatomy, a location of placement of the stent may also be recommended to a user.

An additional aspect of the present disclosure involves coregistering IVUS images from the IVUS imaging device with a venogram image from the x-ray imaging device. In this way, the location of the IVUS imaging probe in relation to regions of compression may be determined. As a result, when an IVUS imaging procedure is performed, the corresponding IVUS image frames within a predetermined distance from a venous compression site may be identified to a user. When the IVUS imaging probe is within this predetermined distance, one or more measurement tools may additionally be triggered to acquire metrics relating to the constricted vessel, such as vessel diameter.

In an exemplary aspect of the present disclosure, a system is provided. The system includes a processor circuit configured for communication with an external imaging device, wherein the processor circuit is configured to: receive, from the external imaging device, an image comprising a blood vessel within a patient; determine, using the image, a first location of the blood vessel with a restriction in blood flow caused by compression of the blood vessel by an anatomical structure within the patient and different than the blood vessel; generate a first graphical representation associated with the restriction; output, to a display in communication with the processor circuit, a screen display comprising: the image; and the first graphical representation at the first location of the blood vessel in the image.

In some aspects, the external imaging device comprises an x-ray imaging device, and the image comprises an x-ray image. In some aspects, the processor circuit is configured to determine the first location of the blood vessel with the restriction using a convolutional neural network. In some aspects, the convolutional neural network is trained using a plurality of images with identified restrictions in blood flow caused by the compression of further blood vessels by further anatomical structures. In some aspects, the processor circuit is configured to classify the first location of the blood vessel with the restriction as a first type of restriction or a second type of restriction. In some aspects, the first type of restriction comprises a location of a ligament and the second type of restriction comprises a crossover of the blood vessel and a further blood vessel. In some aspects, the processor circuit is configured to segment anatomy within the image. In some aspects, the processor circuit is configured to: divide the image into a plurality of patches, wherein each patch of the plurality of patches comprises a plurality of pixels of the image; and determine a patch as the first location of the blood vessel with the restriction. In some aspects, the image comprises a first image, the processor circuit is configured to receive a second image comprising at least one of the blood vessel or the anatomical structure, and the processor circuit is configured to determine the first location of the blood vessel with the restriction using the first image and second image. In some aspects, the first image comprises a first x-ray image obtained with contrast within the blood vessel, and the second image comprises a second x-ray image obtained without contrast within the blood vessel. In some aspects, the first image comprises an x-ray image, the second image comprises an intravascular ultrasound (IVUS) image, the processor circuit is configured for communication with an IVUS catheter, the processor circuit is configured to receive the IVUS image from the IVUS catheter. In some aspects, the first graphical representation comprises a color-coded map corresponding to a severity of the restriction in the blood flow. In some aspects, the processor circuit is configured to: determine a stent recommendation to treat the restriction based on at least one of the image or the first location of the blood vessel with the restriction; and output the stent recommendation to the display. In some aspects, the processor circuit is configured to: determine a stent landing zone at a second location of the blood vessel based on at least one of the stent recommendation, the image, or the first location of the blood vessel with the restriction; generate a second graphical representation of the stent landing zone; and output the second graphical representation at the second location of the blood vessel in the image. In some aspects, the processor circuit is configured to: determine a stent strength position at a third location of the blood vessel based on at least one of the stent landing zone, the stent recommendation, the image, or the first location of the blood vessel with the restriction; generate a third graphical representation of the stent strength position; and output the third graphical representation at the third location of the blood vessel in the image. In some aspects, the processor circuit is configured for communication with an intravascular ultrasound (IVUS) catheter, and the processor circuit is configured to: receive a plurality of IVUS images along a length of the blood vessel from the IVUS catheter, co-register the plurality of IVUS images with the image; identify an IVUS image of the plurality of IVUS image corresponding to the first location of the blood vessel with a restriction; and output the IVUS image to the display.

In an exemplary aspect of the present disclosure, a blood vessel compression identification system is provided. The system includes an x-ray imaging device configured to obtain an x-ray image comprising a vein within a patient; and a processor circuit in communication with the x-ray imaging device, wherein the processor circuit is configured to: receive the x-ray image from the x-ray imaging device; determine, using a deep learning algorithm, a first location of the vein with a restriction in blood flow caused by compression of the vein by an anatomical structure within the patient and different than the vein, wherein the anatomical structure comprises an artery or a ligament; determine a stent recommendation to treat the restriction based on at least one of the x-ray image or the first location of the vein with the restriction; determine a stent landing zone at a second location of the vein based on at least one of the stent recommendation, the x-ray image, or the first location of the vein with the restriction; output, to a display in communication with the processor circuit, a screen display comprising: the x-ray image; a first graphical representation of the stent recommendation; and
a second graphical representation of the stent landing zone overlaid on the x-ray image at the second location of the vein.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging and x-ray system, according to aspects of the present disclosure.

FIG. 2 is a schematic diagram of a processor circuit, according to aspects of the present disclosure.

FIG. 3 is a diagrammatic view of an example anatomy, according to aspects of the present disclosure.

FIG. 4A is a diagrammatic view of an x-ray venogram image of an anatomy with a region of stenosis before treatment, according to aspects of the present disclosure.

FIG. 4B is a diagrammatic view of an x-ray venogram image of an anatomy after an initial treatment, according to aspects of the present disclosure.

FIG. 4C is a diagrammatic view of an x-ray venogram image of an anatomy after placement of a stent, according to aspects of the present disclosure.

FIG. 5 is a schematic diagram of a deep learning network configuration, according to aspects of the present disclosure.

FIG. 6 is a flow diagram of a method of training a deep learning network to identify regions of interest within an x-ray venogram image, according to aspects of the present disclosure.

FIG. 7A is a diagrammatic view of an annotated x-ray venogram image identifying a predicted location of an inguinal ligament, according to aspects of the present disclosure.

FIG. 7B is a diagrammatic view of an annotated x-ray venogram image identifying a predicted crossover location of an iliac vein with an iliac artery, according to aspects of the present disclosure.

FIG. 7C is a diagrammatic view of an annotated x-ray venogram image identifying a predicted location of vein constriction, according to aspects of the present disclosure.

FIG. 7D is a diagrammatic view of an annotated x-ray venogram image identifying anatomical landmarks, according to aspects of the present disclosure.

FIG. 8 is a flow diagram of a method of identifying regions of interest within an x-ray venogram image with a deep learning network, according to aspects of the present disclosure.

FIG. 9 is a schematic diagram for identification of regions of interest within an x-ray venogram image, according to aspects of the present disclosure.

FIG. 10 is a diagrammatic view of a segmented x-ray venogram image identifying regions of interest, according to aspects of the present disclosure.

FIG. 11 is a diagrammatic view of an x-ray venogram image identifying regions of interest, according to aspects of the present disclosure.

FIG. 12 is a flow diagram of a method of identifying IVUS images at locations where an IVUS imaging probe is at or near an anatomical landmark, according to aspects of the present disclosure.

FIG. 13A is a diagrammatic view of a graphical user interface displaying an IVUS image at a location where the IVUS imaging probe is not near an anatomical landmark, according to aspects of the present disclosure.

FIG. 13B is a diagrammatic view of a graphical user interface displaying an IVUS image at a location where the IVUS imaging probe is near an anatomical landmark, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

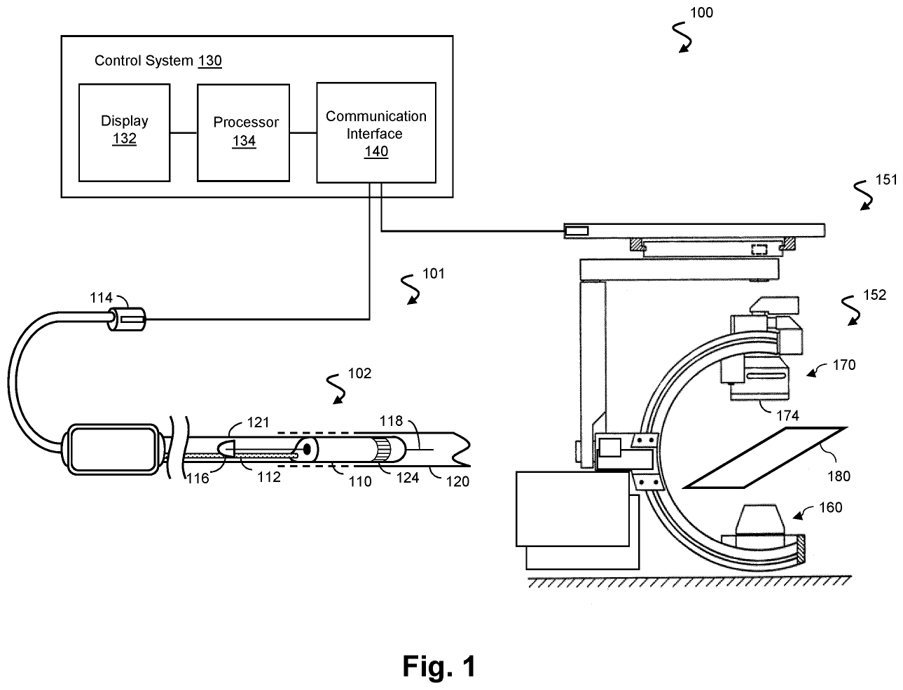

FIG. 1 is a schematic diagram of an intraluminal imaging and x-ray system 100, according to aspects of the present disclosure. In some embodiments, the intraluminal imaging and x-ray system 100 may include two separate systems: an intraluminal ultrasound imaging system 101 and an x-ray imaging system 151. For example, the intraluminal ultrasound imaging system 101 may be in communication with the x-ray imaging system 151 through any suitable means. Such communication may be established through a wired cable, through a wireless signal, or by any other means. In addition, the intraluminal imaging system 101 may be in continuous communication with the x-ray system 151 or may be in intermittent communication. For example, the two systems may be brought into temporary communication via a wired cable, or brought into communication via a wireless communication, or through any other suitable means at some point before, after, or during an examination. In addition, the intraluminal system 101 may receive data such as x-ray images, annotated x-ray images, metrics calculated with the x-ray imaging system 151, information regarding dates and times of examinations, types and/or severity of patient conditions or diagnoses, patient history or other patient information, or any suitable data or information from the x-ray imaging system 151. The x-ray imaging system 151 may also receive any of these data from the intraluminal imaging device 101. In some embodiments, and as shown in FIG. 1, the intraluminal imaging device 101 and the x-ray imaging device 151 may be in communication with the same control system 130. In this embodiment, both systems may be communication with the same display 132, processor 134, and communication interface 140 shown as well as in communication with any other components implemented within the control system 130.

The intraluminal imaging system 101 can be an ultrasound imaging system. In some instances, the intraluminal imaging system 101 can be an intravascular ultrasound (IVUS) imaging system. The intraluminal imaging system 101 may include an intraluminal imaging device 102, such as a catheter, guide wire, or guide catheter, in communication with the control system 130. The control system 130 may include a display 132, a processor 134, and a communication interface 140 among other components. The intraluminal imaging device 102 can be an ultrasound imaging device. In some instances, the device 102 can be an IVUS imaging device, such as a solid-state IVUS device.

At a high level, the IVUS device 102 emits ultrasonic energy from a transducer array 124 included in a scanner assembly or probe 110, also referred to as an IVUS imaging assembly, mounted near a distal end of the catheter device. In some embodiments, the probe 110 can be an intra-body probe, such as a catheter, a transesophageal echocardiography (TEE) probe, and/or any other suitable an endo-cavity probe. The ultrasonic energy is reflected by tissue structures in the surrounding medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The communication interface 140 transfers the received echo signals to the processor 134 of the control system 130 where the ultrasound image (including flow information in some embodiments) is reconstructed and displayed on the display 132. The control system 130, including the processor 134, can be operable to facilitate the features of the IVUS imaging system 101 described herein. For example, the processor 134 can execute computer readable instructions stored on the non-transitory tangible computer readable medium.

The communication interface 140 facilitates communication of signals between the control system 130 and the scanner assembly 110 included in the IVUS device 102. This communication includes the steps of: (1) providing commands to integrated circuit controller chip(s) included in the scanner assembly 110 to select the particular transducer array element(s), or acoustic element(s), to be used for transmit and receive, (2) providing the transmit trigger signals to the integrated circuit controller chip(s) included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) of the scanner assembly 110. In some embodiments, the communication interface 140 performs preliminary processing of the echo data prior to relaying the data to the processor 134. In examples of such embodiments, the communication interface 140 performs amplification, filtering, and/or aggregating of the data. In an embodiment, the communication interface 140 also supplies high- and low-voltage DC power to support operation of the device 102 including circuitry within the scanner assembly 110.

The processor 134 receives the echo data from the scanner assembly 110 by way of the communication interface 140 and processes the data to reconstruct an image of the tissue structures in the medium surrounding the scanner assembly 110. The processor 134 outputs image data such that an image of the vessel 120, such as a cross-sectional image of the vessel 120, is displayed on the display 132. The vessel 120 may represent fluid filled or surrounded structures, both natural and man-made. The vessel 120 may be within a body of a patient. The vessel 120 may be a blood vessel, such as an artery or a vein of a patient's vascular system, including cardiac vasculature, peripheral vasculature, neural vasculature, renal vasculature, and/or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors. It is understood that any suitable gauge wire can be used for the conductors. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a patient interface module (PIM) connector 114 at a proximal end of the device 102. The PIM connector 114 electrically couples the transmission line bundle 112 to the communication interface 140 and physically couples the IVUS device 102 to the communication interface 140. In some embodiments, the communication interface 140 may be a PIM. In an embodiment, the IVUS device 102 further includes a guide wire exit port 116. Accordingly, in some instances the IVUS device 102 is a rapid-exchange catheter. The guide wire exit port 116 allows a guide wire 118 to be inserted towards the distal end to direct the device 102 through the vessel 120.

The x-ray imaging system 151 may include an x-ray imaging apparatus or device 152 configured to perform x-ray imaging, angiography, fluoroscopy, radiography, among other imaging techniques. The x-ray imaging device 152 may be of any suitable type, for example, it may be a stationary x-ray system such as a fixed c-arm x-ray device, a mobile c-arm x-ray device, a straight arm x-ray device, or a u-arm device. The x-ray imaging device 152 may additionally be any suitable mobile device. The x-ray imaging device 102 may also be in communication with the control system 130. In some embodiments, the x-ray system 151 may include a digital radiography device or any other suitable device.

The x-ray device 152 as shown in FIG. 1 includes an x-ray source 160 and an x-ray detector 170 including an input screen 174. The x-ray source 160 and the detector 170 may be mounted at a mutual distance. Positioned between the x-ray source 160 and the x-ray detector 170 may be an anatomy of a patient or object 180. For example, the anatomy of the patient (including the vessel 120) can be positioned between the x-ray source 160 and the x-ray detector 170.

The x-ray source 160 may include an x-ray tube adapted to generate x-rays. Some aspects of the x-ray source 160 may include one or more vacuum tubes including a cathode in connection with a negative lead of a high-voltage power source and an anode in connection with a positive lead of the same power source. The cathode of the x-ray source 160 may additionally include a filament. The filament may be of any suitable type or constructed of any suitable material, including tungsten or rhenium tungsten, and may be positioned within a recessed region of the cathode. One function of the cathode may be to expel electrons from the high voltage power source and focus them into a well-defined beam aimed at the anode. The anode may also be constructed of any suitable material and may be configured to create x-radiation from the emitted electrons of the cathode. In addition, the anode may dissipate heat created in the process of generating x-radiation. The anode may be shaped as a beveled disk and, in some embodiments, may be rotated via an electric motor. The cathode and anode of the x-ray source 160 may be housed in an airtight enclosure, sometimes referred to as an envelope.

In some embodiments, the x-ray source 160 may include a radiation object focus which influences the visibility of an image. The radiation object focus may be selected by a user of the system 100 or by a manufacture of the system 100 based on characteristics such as blurring, visibility, heat-dissipating capacity, or other characteristics. In some embodiments, an operator or user of the system 100 may switch between different provided radiation object foci in a point-of-care setting.

The detector 170 may be configured to acquire x-ray images and may include the input screen 174. The input screen 174 may include one or more intensifying screens configured to absorb x-ray energy and convert the energy to light. The light may in turn expose a film. The input screen 174 may be used to convert x-ray energy to light in embodiments in which the film may be more sensitive to light than x-radiation. Different types of intensifying screens within the image intensifier may be selected depending on the region of a patient to be imaged, requirements for image detail and/or patient exposure, or any other factors. Intensifying screens may be constructed of any suitable materials, including barium lead sulfate, barium strontium sulfate, barium fluorochloride, yttrium oxysulfide, or any other suitable material. The input screen 374 may be a fluorescent screen or a film positioned directly adjacent to a fluorescent screen. In some embodiments, the input screen 374 may also include a protective screen to shield circuitry or components within the detector 370 from the surrounding environment. The x-ray detector 370 may additionally be referred to as an x-ray sensor.

The object 180 may be any suitable object to be imaged. In an exemplary embodiment, the object may be the anatomy of a patient. More specifically, the anatomy to be imaged may include chest, abdomen, the pelvic region, neck, legs, head, feet, a region with cardiac vasculature, or a region containing the peripheral vasculature of a patient and may include various anatomical structures such as, but not limited to, organs, tissue, blood vessels and blood, gases, or any other anatomical structures or objects. In other embodiments, the object may be or include man-made structures.

In some embodiments, the x-ray imaging system 151 may be configured to image venogram fluoroscopy images. In such embodiments, a contrast agent or x-ray dye may be introduced to a patient's anatomy before imaging. The contrast agent may also be referred to as a radiocontrast agent, contrast material, contrast dye, or contrast media. The contrast dye may be of any suitable material, chemical, or compound and may be a liquid, powder, paste, tablet, or of any other suitable form. For example, the contrast dye may be iodine-based compounds, barium sulfate compounds, gadolinium-based compounds, or any other suitable compounds. The contrast agent may be used to enhance the visibility of internal fluids or structures within a patient's anatomy. The contrast agent may absorb external x-rays, resulting in decreased exposure on the x-ray detector 170.

When the control system 130 is in communication with the x-ray system 151, the communication interface 140 facilitates communication of signals between the control system 130 and the x-ray device 152. This communication includes providing control commands to the x-ray source 160 and/or the x-ray detector 170 of the x-ray device 152 and receiving data from the x-ray device 152. In some embodiments, the communication interface 140 performs preliminary processing of the x-ray data prior to relaying the data to the processor 134. In examples of such embodiments, the communication interface 140 may perform amplification, filtering, and/or aggregating of the data. In an embodiment, the communication interface 140 also supplies high- and low-voltage DC power to support operation of the device 152 including circuitry within the device.

The processor 134 receives the x-ray data from the x-ray device 152 by way of the communication interface 140 and processes the data to reconstruct an image of the anatomy being imaged. The processor 134 outputs image data such that an image is displayed on the display 132. In an embodiment in which the contrast agent is introduced to the anatomy of a patient and a venogram is to be generated, the particular areas of interest to be imaged may be one or more blood vessels or other section or part of the human vasculature. The contrast agent may identify fluid filled structures, both natural and/or man-made, such as arteries or veins of a patient's vascular system, including cardiac vasculature, peripheral vasculature, neural vasculature, renal vasculature, and/or any other suitable lumen inside the body. For example, the x-ray device 152 may be used to examine any number of anatomical locations and tissue types, including without limitation all of the organs, fluids, or other structures or parts of an anatomy previously mentioned. In addition to natural structures, the x-ray device 152 may be used to examine man-made structures such as any of the previously mentioned structures.

The processor 134 may be configured to receive a venogram fluoroscopy image that was stored by the x-ray imaging device 152 during a clinical procedure. The images may be further enhanced by other information such as patient history, patient record, IVUS imaging, pre-operative ultrasound imaging, pre-operative CT, or any other suitable data.

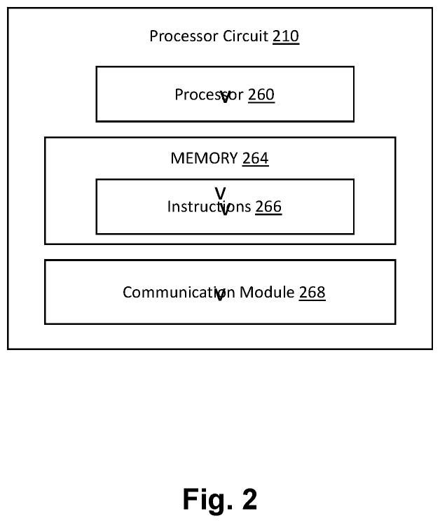

FIG. 2 is a schematic diagram of a processor circuit, according to aspects of the present disclosure. The processor circuit 210 may be implemented in the host system 130 of FIG. 1, the intraluminal imaging system 101, and/or the x-ray imaging system 151, or any other suitable location. In an example, the processor circuit 210 may be in communication with intraluminal imaging device 102, the x-ray imaging device 152, the display 132 within the system 100. The processor circuit 210 may include the processor 134 and/or the communication interface 140 (FIG. 1). One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 760, cause the processor 260 to perform the operations described herein with reference to the probe 110 and/or the host 130 (FIG. 1). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 710, the probe 110, and/or the display 132 and/or display 266. In that regard, the communication module 268 can be an input/output (I/O) device. In some instances, the communication module 268 facilitates direct or indirect communication between various elements of the processor circuit 210 and/or the probe 110 (FIG. 1) and/or the host 130 (FIG. 1).

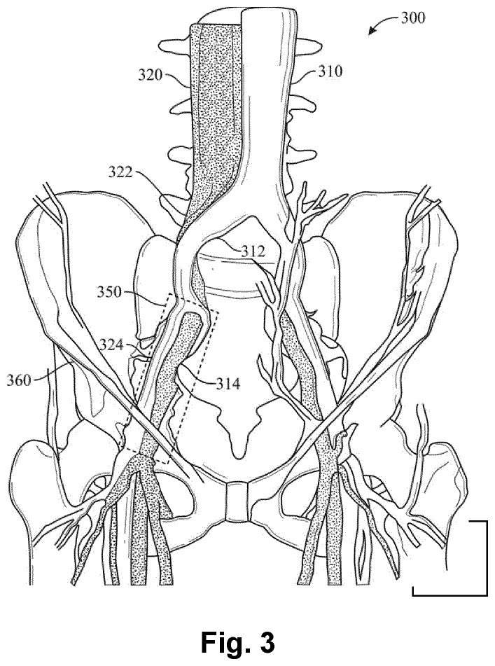

FIG. 3 is a diagrammatic view of an example anatomy 300, according to aspects of the present disclosure. The example anatomy 300 includes the pelvic region, and portions of the abdomen and legs. FIG. 3 may illustrate several regions of likely compression of vessels in a patient's vasculature which the invention of the present disclosure seeks to remedy. For example, compressive venous disease (e.g. May-Thurner) occurs when bones, ligaments, or arteries compress the iliac vein and inhibit venous return. In general, venous compression is caused by a vessel passing through a tight anatomic space due to adjacent structures such as bones, arteries, and/or ligaments as shown in FIG. 3. This leads to restricted cross-sectional area of the vessel and restricted blood flow. Venous compression experienced by a patient may be one or multiple of several venous compression syndromes, including Paget-Schroetter syndrome, Nutcracker syndrome, May-Thurner syndrome, and popliteal venous compression, among others. Unlike other vascular diseases, these syndromes are usually seen in young, otherwise healthy individuals and can lead to significant overall morbidity. FIG. 3 includes a depiction of an abdominal aorta 310, an inferior vena cava 320, a common iliac artery 312, a common iliac vein 322, an external iliac artery 324, an external iliac vein 314, an inguinal ligament 360, and a region 350 corresponding to an area of likely crossover of the external iliac artery 324 and the external iliac vein 314.

The abdominal aorta 310 is among the largest arteries in the human body and carries oxygenated blood from the heart to the lower peripheral vasculature. The abdominal aorta 310, at a location near the hip, divides into two smaller vessels, the common iliac arteries. The common iliac artery 312 is in connection with the external iliac artery 324 shown in FIG. 3. All of these vessels provide oxygenated blood to various structures within the peripheral vasculature of the body.

Positioned adjacent to external iliac artery 312 is the external iliac vein 324. As shown by region 350, at some location along the external iliac vein 324, the external iliac artery 312 may cross over the external iliac vein 314. In such a configuration, the external iliac artery 324 may compress the external iliac vein 314 on its own or against bone or other structures within the anatomy causing a restriction in blood flow. In some instances, the iliac artery 324 may compress the iliac vein 314 against the spine where it crosses over the iliac vein 314. This restriction may be remedied with the placement of a stent within the external iliac vein 324 but the location of crossover of the external iliac vein 314 and the external iliac artery 324 must be determined. Connected to the external iliac vein 314 is the common iliac vein 322 and the inferior vena cava 320.

An additional common location of venous compression may be at a location at or near the inguinal ligament 360. In some cases, the inguinal ligament 360, like the external iliac artery 324, may compress the external iliac vein 314 and inhibit blood flow. Again, the positioning of a stent may help to combat this compression and restore blood flow but the location of the inguinal ligament 360 must be known.

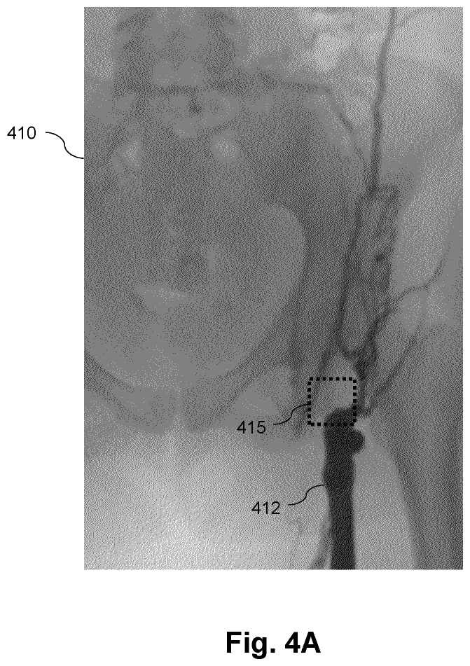

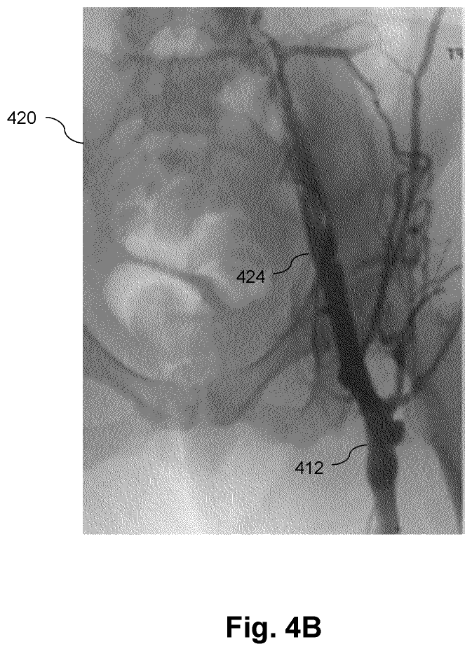

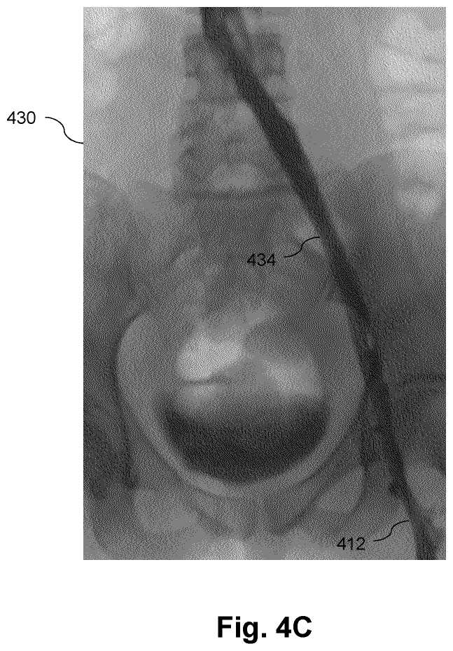

FIGS. 4A, 4B, and 4C illustrate the effects of medical treatments to regions of blood flow restriction in the peripheral vasculature. For example, FIG. 4A is a diagrammatic view of an x-ray venogram image 410 of an anatomy with a region of blood flow restriction 415 before treatment, according to aspects of the present disclosure. FIG. 4A depicts an x-ray venogram image 410, the iliac vein 412, and the region of blood flow restriction 415.

As shown in FIG. 4A, the diameter of the iliac vein 412 at the region 415 is dramatically reduced. An increased amount of blood may also be seen in the lower regions of the iliac vein 412 below the constriction point or region of stenosis 415 because blood flow from the lower part of the vessel is restricted in its return to the heart. The blood shown within the vasculature in FIG. 4A may be more visible than other regions of the x-ray image 410 due to a contrast agent.

The region of blood flow restriction 415 may be of any suitable type or may be caused by any suitable condition. For example, the region of stenosis 415 may be caused by compression type conditions such as compression caused by the inguinal ligament 360 (FIG. 3), the crossover of the iliac artery 324 with the iliac vein 314, or any other physical compression of the iliac vein 324. In addition, the region of blood flow restriction 415 may be caused by thrombus or plaque build-up within the iliac vein 412 itself. This condition may be caused by deep vein thrombosis (DVT) or any other similar condition.

Although. FIGS. 4A, 4B, and 4C, among other figures disclosed in the present application, primarily depict anatomy surrounding the iliac vein and although the present disclosure primarily describes stenosis in the iliac vein, the systems, devices, and methods of the present disclosure may be readily applied to any suitable vein or artery in a patient's anatomy. For example, the venogram depicted in FIG. 4A in another embodiment need not be a venogram but could alternatively be an angiography image, fluoroscopy image, computed tomography (CT) angiogram, CT venogram, or any other suitable image. Additionally, the constricted vein shown may alternatively be an artery, or any vessel (artery or vein) within the heart, leg, arm, abdomen, neck, brain, head or any suitable vessel within the body. In such embodiments, any suitable physical structures within a patient anatomy may be a cause of stenosis and the systems, devices, and methods described herein may be configured to identify these different physical structures accordingly.

FIG. 4B is a diagrammatic view of an x-ray venogram image 420 of an anatomy after an initial treatment, according to aspects of the present disclosure. FIG. 4B depicts an x-ray venogram image 420, the same region of the iliac vein 412, and upper portion 424 of the iliac vein 412.

The x-ray venogram image 420 shown in FIG. 4B may be an image of the anatomy of the same patient shown in FIG. 4A. A number of treatment options are available to treat regions of blood flow restriction within a patient. For example, if the vein has a stenosis (e.g., in region 415 in FIG. 4A), the blood flow restriction can be treated with catheter-direct infusion, angioplasty, medication, bypass, other surgery, or other forms of treatment. FIG. 4B may represent a blockage site after treatment with catheter-direct infusion of a pharmacological agent. As shown by the at least partially restored blood flow of the upper region 424 of the iliac vein 412, the diameter of the vein lumen has been at least partially increased as a result of, e.g., the pharmacological agent breaking down the plaque or thrombus build up in the region 415 of FIG. 4A. In addition, in some cases, the diameter of the iliac vein 412 below the previous location of the region of the stenosis (FIG. 4A) may also be reduced indicating increased blood flow and less stagnation.

FIG. 4C is a diagrammatic view of an x-ray venogram image 430 of an anatomy after placement of a stent, according to aspects of the present disclosure. FIG. 4C depicts an x-ray venogram image 430, the same region of the iliac vein 412, and a upper portion 434 of the iliac vein 412.

The x-ray venogram image 430 shown in FIG. 4C may be an image of the anatomy of the same patient shown in FIG. 4A. In some cases, some forms of treatment, such as angioplasty or other treatments, may cause lesions that can be highly fibrotic, which may result in further vessel compression or blockage. Stenting a blocked or compressed vessel is one way to reduce fibrotic lesions and help reduce the risk of restenosis. In cases where a region of stenosis is observed at or near the inguinal ligament 360, or at the location 350 where the iliac artery 324 and the iliac vein 314 cross over (FIG. 3), a stent may be placed above the profunda femoral veins confluence and into the common femoral vein. The stent may be of any suitable type, such as a Wallstent™ from Boston Scientific, a VICI® stent from Boston Scientific, a Zilver® Vena™ stent from Cook, a Sinus-Venous stent by Optimed, a Venovo® stent by Bard, an ABRE® stent from Medtronic, or any other suitable stent. Any stent that is flexible, is available in large-diameter sizes, and has fracture resistance may be a suitable stent used in the present invention, as will be described in more detail hereafter.

FIG. 4C may represent a blood flow restriction site after positioning a stent within the iliac vein 412. As shown by the more fully restored blood flow of the upper region 434 of the iliac vein 412, the procedure may result in more fully increased diameter of the vein lumen. In addition, in some cases, the diameter of the iliac vein 412 below the previous location of the region of the stenosis (FIG. 4A) may also be reduced indicating increased blood flow and less stagnation. In some cases, the placement of a stent in addition to the angioplasty treatment or other treatment performed in relation to FIG. 4B may additionally increase the blood flow through the iliac vein 412 and result in a decreased likelihood of restenosis.

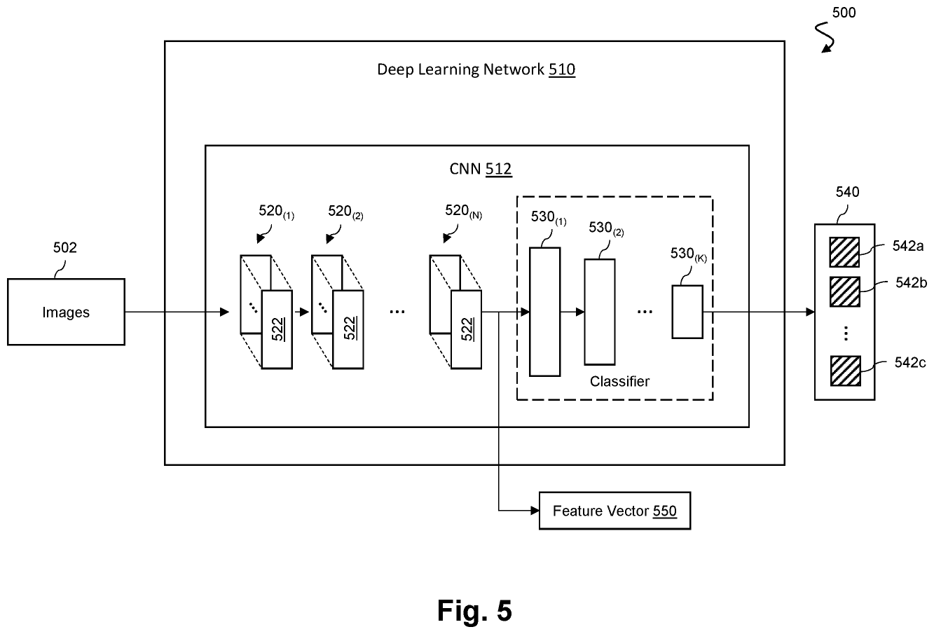

FIG. 5 is a schematic diagram of a deep learning network configuration 500, according to aspects of the present disclosure. The configuration 500 can be implemented by a deep learning network. The configuration 500 includes a deep learning network 510 including one or more CNNs 512. For simplicity of illustration and discussion, FIG. 5 illustrates one CNN 512. However, the embodiments can be scaled to include any suitable number of CNNs 512 (e.g., about 2, 3 or more). The configuration 500 can be trained for identification of various anatomical landmarks or features within a patient anatomy, including a region of crossover of an iliac artery with an iliac vein, pelvic bone notches or other anatomical landmarks or features which may be used to identify the location of an inguinal ligament, and/or other regions of blood flow restriction (e.g., stenosis or compression) as described in greater detail below.

The CNN 512 may include a set of N convolutional layers 520 followed by a set of K fully connected layers 530, where N and K may be any positive integers. The convolutional layers 520 are shown as 520(1) to 520(N). The fully connected layers 530 are shown as 530(1) to 530(K). Each convolutional layer 520 may include a set of filters 522 configured to extract features from an input 502 (e.g., x-ray venogram images or other additional data). The values N and K and the size of the filters 522 may vary depending on the embodiments. In some instances, the convolutional layers 520(1) to 520(N) and the fully connected layers 530(1) to 530(K−1) may utilize a leaky rectified non-linear (ReLU) activation function and/or batch normalization. The fully connected layers 530 may be non-linear and may gradually shrink the high-dimensional output to a dimension of the prediction result (e.g., the classification output 540). Thus, the fully connected layers 530 may also be referred to as a classifier. In some embodiments, the fully convolutional layers 520 may additionally be referred to as perception or perceptive layers.

The classification output 540 may indicate a confidence score for each class 542 based on the input image 502. The classes 542 are shown as 542a, 542b, . . . , 542c. When the CNN 512 is trained for regions of stenosis or general venous compression, the classes 542 may indicate an inguinal ligament class 542a, a crossover class 542b, a pelvic bone notch class 542c, a region of blood flow restriction class 542d, or any other suitable class. A class 542 indicating a high confidence score indicates that the input image 502 or a section or pixel of the image 502 is likely to include an anatomical object/feature of the class 542. Conversely, a class 542 indicating a low confidence score indicates that the input image 502 or a section or pixel of the image 502 is unlikely to include an anatomical object/feature of the class 542.

The CNN 512 can also output a feature vector 550 at the output of the last convolutional layer 520(N). The feature vector 550 may indicate objects detected from the input image 502 or other data. For example, the feature vector 550 may indicate a region of crossover of an iliac artery with an iliac vein, pelvic bone notches or other anatomical landmarks or features which may be used to identify the location of an inguinal ligament, pubic tubercle, anterior superior iliac spine, superior pelvic ramus and/or other regions of blood flow restriction (e.g., stenosis or compression) identified from the image 502.

The deep learning network 510 may implement or include any suitable type of learning network. For example, in some embodiments and as described in relation to FIG. 5, the deep learning network 510 could include a convolutional neural network 512. In addition, the convolutional neural network 510 may additionally or alternatively be or include a multi-class classification network, an encoder-decoder type network, or any suitable network or means of identifying features within an image.

In an embodiment in which the deep learning network 510 includes an encoder-decoder network, the network may include two paths. One path may be a contracting path, in which a large image, such as the image 502, may be convolved by several convolutional layers 520 such that the size of the image 502 changes in depth of the network. The image 502 may then be represented in a low dimensional space, or a flattened space. From this flattened space, an additional path may expand the flattened space to the original size of the image 502. In some embodiments, the encoder-decoder network implemented may also be referred to as a principal component analysis (PCA) method. In some embodiments, the encoder-decoder network may segment the image 502 into patches.

In an additional embodiment of the present disclosure, the deep learning network 510 may include a multi-class classification network. In such an embodiment, the multi-class classification network may include an encoder path. For example, the image 502 may be of a high dimensional image. The image 502 may then be processed with the convolutional layers 520 such that the size is reduced. The resulting low dimensional representation of the image 502 may be used to generate the feature vector 550 shown in FIG. 5. The low dimensional representation of the image 502 may additionally be used by the fully connected layers 530 to regress and output one or more classes 542. In some regards, the fully connected layers 530 may process the output of the encoder or convolutional layers 520. The fully connected layers 530 may additionally be referred to as task layers or regression layers, among other terms.

Any suitable combination or variations of the deep learning network 510 described is fully contemplated. For example, the deep learning network may include fully convolutional networks or layers or fully connected networks or layers or a combination of the two. In addition, the deep learning network may include a multi-class classification network, an encoder-decoder network, or a combination of the two.

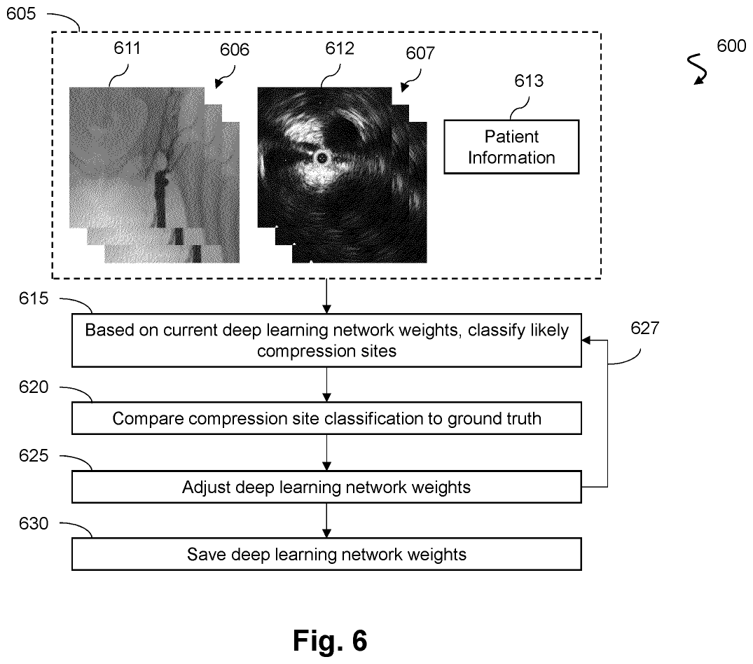

FIG. 6 is a flow diagram of a method 600 of training a deep learning network 510 to identify regions of interest within an x-ray venogram image, according to aspects of the present disclosure. One or more steps of the method 600 can be performed by a processor circuit of the system 100, including, e.g., the processor 134 (FIG. 1). As illustrated, the method 600 includes a number of enumerated steps, but embodiments of the method 600 may include additional steps before, after, or in between the enumerated steps. In some embodiments, one or more of the enumerated steps may be omitted, performed in a different order, or performed concurrently. The deep learning network may be trained with any suitable method or approach such as any gradient descent, stochastic, batch, mini-batch approach, or any other optimization algorithm, method, or approach. In an embodiment, the deep learning network may be trained using a mini-batch approach.

At step 605, the method 600 includes receiving various input images and/or data to the deep learning network 510. Various forms or types of data may be input into the deep learning network 510. For example, an x-ray venogram image 611, one or more IVUS images 612, as well as other patient information 613 may be included as inputs to the deep learning network 510 either during a training process as described, or during implementation of the deep learning network 510 to identify compression sites within the anatomy of patient.

During training, multiple x-ray venogram images 611 may be input to the deep learning network 510. The venogram images 611 may depict any of the previously mentioned likely compression sites or locations of restrictions of blood flow in a vessel, including the inguinal ligament, a region of crossover of the iliac artery with the iliac vein, other general regions of stenosis, or other regions of interest, such as notches of the pelvic bone. The locations of the notches in the pelvic bone may correspond to the location of the inguinal ligament which may not be visible in angiography images. For example, the inguinal ligament can extend between the notches. For training, the venogram images 611 may be annotated by experts in the field to identify some or all of these features. In some embodiments, each expert may examine each image 611 and highlights or otherwise identify pixels, segments, or patches that demark the location of the inguinal ligament, the crossover of the iliac artery and the iliac vein, the notches of the pelvic bone, or other regions of interest that may denote venous compression. In some embodiments, experts may additionally identify or rate the severity of the compression sites. These annotated venogram images 611 may serve as ground truth data during a training of the deep learning network 510. The annotated venogram images 611 that are used to train the deep learning network 510 may collectively be referred to as a training data set or training set 606. The training data set 606 may be generated from any suitable number of unique x-ray venogram images from many different patients. For example, the training data set 606 may include 5, 10, 15, 20, 30, 60, 100, or more unique x-ray venogram images, as well as any number therebetween. In some embodiments, more than 30 unique images acquired from different patients undergoing venous stenting in the iliac region may be included in a training data set 606 of x-ray venogram images 611. In some embodiments, annotations from experts in the field may be embedded within x-ray venogram images 611 to form one uniform image or image file. The annotations may include data representations within or associated with an image file. The annotations may also include graphical representations such various colors, patterns, shapes, highlights, arrows, indicators, or any other suitable graphical representation to denote any of the compression sites, their types and/or severity as needed. In other embodiments, annotations from experts may be saved as separate files from the x-ray venogram images. For example, a mask including expert annotations may be stored in conjunction with the venogram images 611 as the ground truth.

An additional input to the deep learning network 510 may include IVUS images 612 that are co-registered with the annotated venogram images 611. In some embodiments, co-registration of IVUS images 612 with a venogram image 611 may allow a user or the system 100 to identify an association of IVUS images 612 imaged at locations near determined anatomical landmarks within a venogram image 611. The coregistration of IVUS images 612 with venogram images 611 in the present disclosure may share some similar aspects or features of coregistering data from different devices as those disclosed in U.S. Pat. No. 6,428,930, which is hereby incorporated by reference in its entirety. Various metrics may be provided by IVUS images 612 to the deep learning network 510 including but not limited to a vessel diameter, vessel area, lumen diameter, lumen area, locations of blockages within a vessel, the size of such blockages, the severity of blood flow restriction, among others. This data may then be used as an additional input by the deep learning network to more accurately identify any of the previously mentioned compression sites. In some embodiments, input IVUS images 612 may be used to identify regions of blood flow restriction, and/or the locations of neighboring blood vessels or ligaments (e.g., the location of an artery next to a vein, the location of the inguinal ligament next to a blood vessel). Input IVUS images 612 may additionally be organized into a set 607. There may be any suitable number of IVUS images 612 within the set 607 including any of the numbers of input venogram images 611.

Additional input images are also contemplated. For example, x-ray images that do not involve fluoroscopy may be used to aid the deep learning network 510 to more accurately identify the mentioned compression sites. Other ultrasound images, CT images, magnetic resonance imaging (MM) images or any other suitable images from other imaging techniques may be input to train the deep learning network 510.

The additional patient information 613 may also serve as an input to the deep learning network. For example, additional patient information 613 may include patient history including past diagnoses, past locations of stenosis, stents, the success of various treatments in remedying regions of stenosis, other patient information including patient trends such as weight, age, height, systolic and/or pulse blood pressure, blood type, or other information regarding patient conditions or any other data or information. With additional patient information 613 an additional input, the deep learning network may more accurately identify areas of venous compression.

At step 615, the method 600 includes classifying likely compression sites based on current deep learning network weights. Deep learning network weights may represent the strength of connections between units in adjacent network layers. In some embodiments, the linear transformation of network weights and the values in the previous layer passes through a non-linear activation function to produce the values of the next layer. This process may happen at each layer of the network during forward propagation. The deep learning network weights may be additionally or alternatively referred to as coefficients, filters, or parameters, among other terms.

In some embodiments, the deep learning network may analyze an x-ray venogram image 611 and classify either the image as a whole, segments or patches of the image, or pixels of the image as any of the previously mentioned classes. For example, for a given segment or patch of an image 611, the deep learning network may classify the segment or patch as the inguinal ligament class 542a (FIG. 5) if it determines that the inguinal ligament is likely present in the image segment or patch. As an additional example, for a given segment or patch of an image 611, the deep learning network may classify the segment or patch as a region of crossover of the iliac artery and the iliac vein, or class 542b (FIG. 5) if it determines that the iliac artery crosses over the iliac vein at that image segment or patch. In some embodiments, each output class 542 may be identified through separate binaries. In other embodiments, one multi-class classification network may be trained and implemented to identify different classes 542 (FIG. 5).

At step 620, the method 600 includes comparing compression site classification outputs from the deep learning network to the ground truth annotated x-ray venogram images. When the deep learning network has classified the image 611 into any of the various classes 542 (FIG. 5) it is to be trained to identify, the output may be compared to same x-ray venogram image 611 annotated by experts. In some embodiments, a degree of error is calculated for each output classification representing the difference between the deep learning network's output and the annotated image. In some embodiments, a loss function may be used to determine the degree of error for each classification. In some embodiments, the loss function may include a cross-entropy loss function, or log loss function, or any other suitable means of evaluating the accuracy of the deep learning network output may be used at step 620.

At step 625, the method 600 includes adjusting the deep learning network weights to more accurately identify likely compression sites. Based on the degree of error calculated for each class 542 (FIG. 5), the deep learning network weights may be adjusted. As shown by an arrow 627 shown in FIG. 6, the method 600 may then revert back to step 615 and the process of classifying images 611 or segments of images 611 may begin again. As steps 615, 620, and 625 are iteratively performed, the degree of error calculated for each class 542 may progressively decrease until all of the x-ray venogram images 611 have been presented to the deep learning network. In other words, at each iteration in the training, a batch of the images 611 from the training data set 606 is processed and the weights of the networks are optimized so the predictions of likely compression sites generate low error at the output. In some embodiments, a back propagation algorithm may be used to optimize the weights of the deep learning network. For example, the network may back propagate the errors to update the weights.

At step 630, the method 600 includes saving the deep learning network weights as a deep learning network file. After all the x-ray venogram images and other inputs, optionally including coregistered IVUS images 612 and other patient information 613 has been input and processed by the deep learning network and the deep learning network weights have been adjusted, a file may be created and stored corresponding to the deep learning network. This file may be subsequently loaded by the system 100 when performing patient examinations of similar regions of anatomies to assist a user of the system 100 to identify likely compression sites.

In some embodiments, multiple deep learning networks may be trained. For example, one deep learning network may be trained based on venogram images 611 and another network may be trained on IVUS images 612. Any one or combination of these deep learning networks may trained and/or implemented as described herein

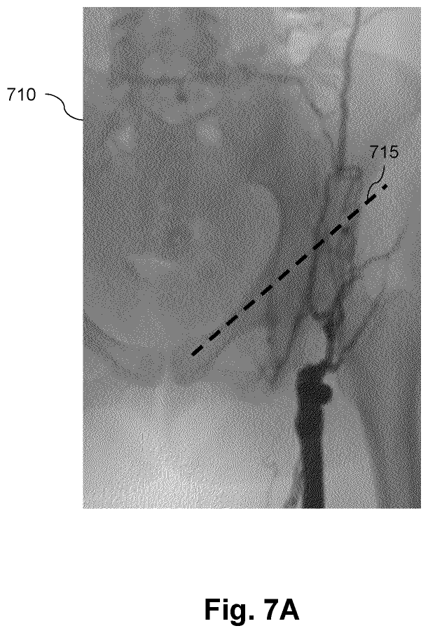

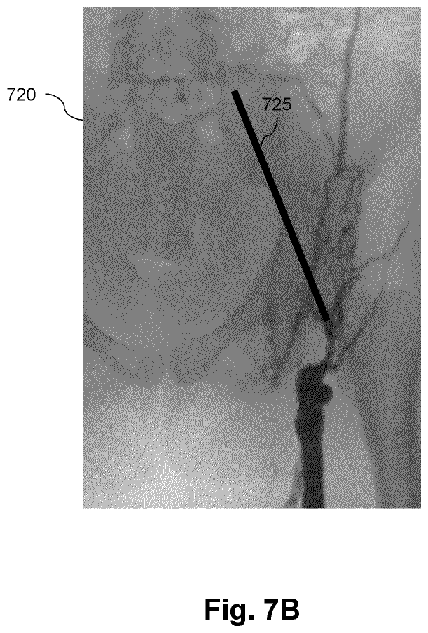

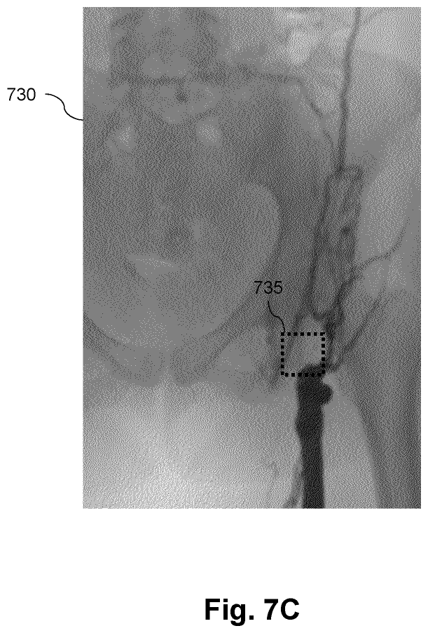

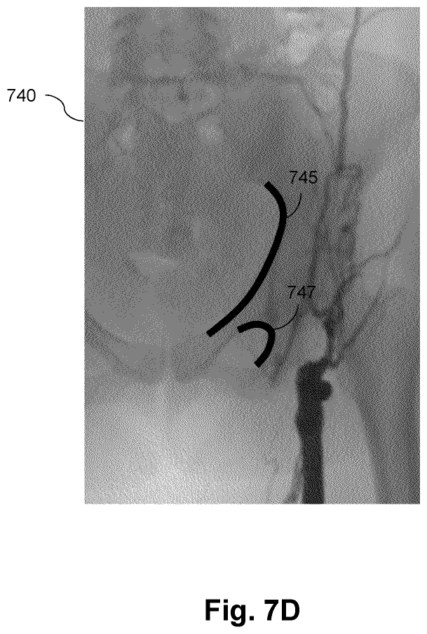

FIG. 7A is a diagrammatic view of an annotated x-ray venogram image 710 identifying a predicted location of an inguinal ligament, according to aspects of the present disclosure. Image 710 may be an annotated image 611 of the training data set 606 of FIG. 6 or it may be an output of the deep learning network in relation to a patient examination. The predicted location of the inguinal ligament may be denoted by any suitable graphical element 715. For example, as shown in FIG. 7A, the graphical element 715 may be a dotted line. In other embodiments, the graphical element 715 identifying the location of the inguinal ligament may be any other graphical representation including a line of any pattern, curve, profile, color, or width, any geometric or non-geometric shape, any indicator such as an arrow, flag, marker, point, any alpha-numerical text, or any other graphical representation. In some embodiments, the graphical element 715 may be overlaid on the image 710 and displayed to a user of the system 100.

FIG. 7B is a diagrammatic view of an annotated x-ray venogram image 720 identifying a predicted crossover location of an iliac vein with an iliac artery, according to aspects of the present disclosure. Similar to image 710, image 720 may be one of the training data set 606 of FIG. 6 or it may be an output of the deep learning network. The predicted region of a crossover of the iliac artery and the iliac vein may be denoted by any suitable graphical element 725. For example, as shown in FIG. 7B, the graphical element 725 may be a solid line. In other embodiments, the graphical element 725 identifying the location of the crossover of the iliac artery and the iliac vein may be any other graphical representation including any of the previously mentioned graphical representations listed corresponding to graphical element 715 of FIG. 7A. The graphical element 725 may be overlaid on the image 720 and displayed to a user of the system 100.

FIG. 7C is a diagrammatic view of an annotated x-ray venogram image 730 identifying a predicted location of vein constriction, according to aspects of the present disclosure. Such a vein construction, as shown by a graphical element 735 overlaid on the image 730, may be caused by physical compression, thrombus, plaque, fibrotic scar tissue buildup, or any other cause. Image 730 may be one of the training data set 606 of FIG. 6 or it may be an output of the deep learning network. The region of stenosis may be denoted by any suitable graphical element 735. For example, as shown in FIG. 7C, the graphical element 735 may be a rectangular shape. In other embodiments, the graphical element 735 may be any other graphical representation including any of the previously mentioned graphical representations listed corresponding to graphical element 715 of FIG. 7A. The graphical element 735 may be overlaid on the image 730 and displayed to a user of the system 100.

FIG. 7D is a diagrammatic view of an annotated x-ray venogram image 740 identifying anatomical landmarks, according to aspects of the present disclosure. Like images 710, 720, and 730, image 740 may be one of the training data set 606 of FIG. 6 or it may be an output of the deep learning network. The anatomical landmarks identified in the image 740 may be any anatomical landmark of interest to the user. For example, in some embodiments, the location of notches within the pelvic bone may be identified as anatomical landmarks to more clearly show the predicted location of the inguinal ligament and predicted compression sites. In some embodiments, the location of the notches of the pelvic bone as identified in FIG. 7D may assist the system 100 and/or the deep learning network 500 in identifying the location of the inguinal ligament. For example, in some embodiments, the output of the deep learning network corresponding to the location of the notches of the pelvic bone may serve as an additional input to determine the location of the inguinal ligament. Thus, in some embodiments, the system 100 and/or the deep learning network 500 can first identify landmarks like notches in the pelvic bone, anterior superior iliac spine, superior pelvic ramus etc. (which are visible in the x-ray image) and then infer the location of the inguinal ligament (which is not visible in the x-ray image). The notches in the pelvic bone are shown identified in FIG. 7D by a graphical element 745 and a graphical element 747. The graphical elements 745 and 747, though seen as solid lines positioned along the edge of the notches of the pelvic bone in FIG. 7, may be any graphical representation including any of the previously mentioned graphical representations listed corresponding to graphical element 715 of FIG. 7A. The graphical elements 745 and 747 may be overlaid on the image 740 and displayed to a user of the system 100. The pelvic notches are one example of anatomical landmarks that can be identified. The deep learning network can additionally identify other anatomical landmarks including the pubic tubercle, anterior superior spine, superior pelvic ramus, or any other suitable anatomical landmarks.

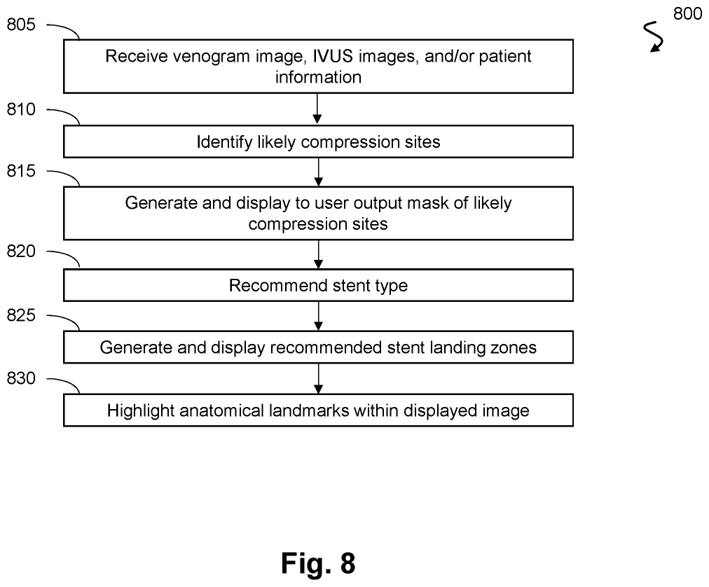

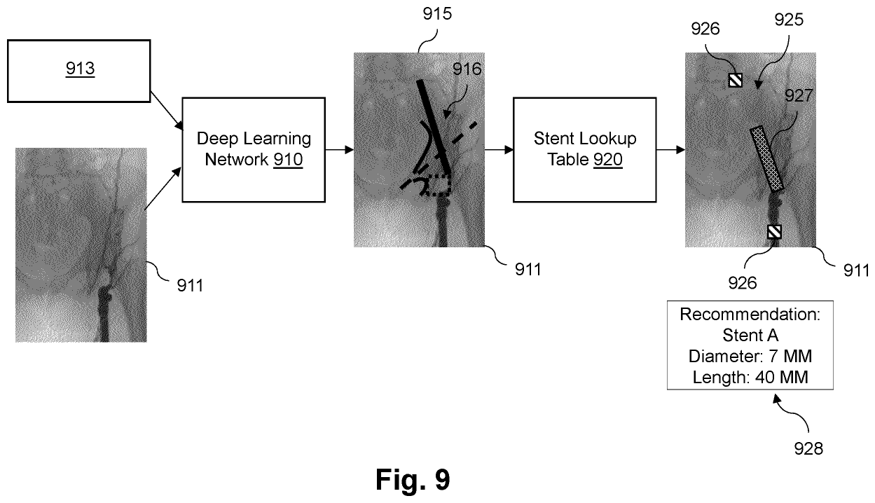

FIG. 8 is a flow diagram of a method 800 of identifying regions of interest within an x-ray venogram image 911 with a deep learning network 910, according to aspects of the present disclosure. One or more steps of the method 800 will be described with reference to FIG. 9, which is a schematic diagram for identification of regions of interest within an x-ray venogram image 911, according to aspects of the present disclosure. One or more steps of the method 800 can be performed by a processor circuit of the system 100, including, e.g., the processor 134 (FIG. 1). As illustrated, the method 800 includes a number of enumerated steps, but embodiments of the method 800 may include additional steps before, after, or in between the enumerated steps. In some embodiments, one or more of the enumerated steps may be omitted, performed in a different order, or performed concurrently.

At step 805, the method 800 includes receiving one or more venogram images 911, one or more IVUS images, and/or patient information 913. Any of the same forms of data that were received at step 605 of the training method 600 (FIG. 6) of the deep learning network may be received as inputs during an implementation of the network 910. While the venogram images 611, IVUS images 612 and other information 613 received at step 605 of the method 600 may be annotated by an expert and used to train the deep learning network, the venogram images 911, IVUS images, and/or other patient information 913 received at step 805 are not expert annotated and correspond to an implementation of the deep learning network 911 which has been previously trained. For example, the venogram images 911 and other inputs 913 may correspond to a patient with a venous compression disorder and the deep learning network 910 may assist a physician in identifying likely compression sites. Any suitable number of images 911 or other data 913 may be received at step 605. For example, in some embodiments, the deep learning network 910 may receive a single x-ray venogram image 911 of the anatomy of a patient. In other embodiments, the deep learning network 910 may receive a single x-ray venogram image 911 with one co-registered IVUS image, a single venogram image 911 with multiple co-registered IVUS images, multiple venogram images 911, any other possible input data 913 such as other patient information previously mentioned or a combination of all of these. The venogram images 911 or IVUS images may in some cases depict regions of venous compression. The venogram images 911 received may be x-ray angiography images acquired with a contrast agent introduced to the patient anatomy, or x-ray fluoroscopy images acquired without a contrast agent introduced to the patient anatomy. In some embodiments, the system 100 may receive one angiography image 911 with contrast and one fluoroscopy image 911 without contrast as inputs. In some embodiments, the received venogram images 911 may depict a blood vessel with a restriction of blood flow. This restriction of blood flow may be caused by compression from an anatomical structure in the anatomy, including any of the structures previously described. In some embodiments, the anatomical structure may be visible within the received venogram images 911 or may not be visible. In some embodiments, other anatomical structures that are visible within the venogram images 911 may assist a physician, or the system 100 as will be described in more detail, to identify an anatomical structure causing a restriction in blood flow in the vessel that is not visible in the received venogram images 911.

At step 810, the method 800 includes identifying likely compression sites. The received inputs, including venogram images 911, IVUS images, and/or other patient information 913, may be processed through the layers of the deep learning network to sort the images 911 or segments of images 911 into classes 542 (FIG. 5). The deep learning network 910 may be substantially similar to that disclosed in FIG. 5 and any of the previously mentioned types of network elements may be employed. In some embodiments, the deep learning network 910 may generate a confidence score for an input image 911 relating to each class it is trained to identify. The confidence score may be of any suitable type or range. For example, a confidence score for a given class 542 (FIG. 5) may be a numeral between the values of 0 and 1, 0 corresponding to an image that does not show any features indicative of the class 542 and 1 corresponding to an image that does show one or more features indicative of the class 542 and a maximum confidence of identification of features of the class 542. Any numeral between the numerals 0 and 1 may represent some confidence level less than the maximum confidence represented by a score of 1, but more than the minimum score of 0. Any suitable numbers may be used to define the range of possible confidence scores. In addition, any suitable method of calculating the likelihood of the presence of a class 542 may be employed by the deep learning network at step 810. In other embodiments, the deep learning network may divide a received input into segments or patches and may calculate a confidence score for each segment or patch. In still other embodiments, the deep learning network may assign a confidence score relating to the available classes 542 to each pixel within the received image. In some embodiments, the deep learning network 910, a manufacturer of the system 100, experts in the field, or a user of the system 100 may determine a threshold confidence score level. When the confidence score associated with a particular class 542 (FIG. 5) exceeds this predetermined threshold, the system 100 may identify the class 542 in the image 911 or otherwise indicate the prediction of the class 542. In some embodiments, the system 100 may display to a user the confidence scores associated with each class 542 via the display 132. At step 810, the system 100 may determine the locations of restrictions in flow of blood vessels within the received venogram images 911. The system 100 may identify any suitable number of locations of restrictions of blood flow within the vessels. For example, in some embodiments, the system may identify one, two, three, four, or more locations of restricted blood flow. Each location may be displayed separately, or multiple locations may be displayed together. These locations may be depicted in a single venogram image or in different venogram images. These locations may also be depicted in various IVUS images or other patient information.

At step 815, the method 800 includes generating and displaying to a user an output mask 915 of likely compression sites. The system 100 may display to a user, via the display 132 (FIG. 1), the venogram image 911 input to the deep learning network 910 at step 805 with the output mask including one or more graphical representations corresponding to locations of restrictions of blood flow of the vessels shown. These graphical representations may be displayed at the locations of restriction within the venogram image(s) 911. Depending on the classification of various segments or parts of the image, the output venogram image may appear substantially similar to any of FIGS. 7A, 7B, 7C, or 7D or any combination thereof. In some embodiments, one or more graphical elements 916 may additionally be generated and presented overlaid on the received venogram image 911 as a mask 915. The graphical elements 916 may be substantially similar to the graphical elements 715 (FIG. 7A), 725 (FIG. 7B), 735 (FIG. 7C), 745, and/or 747 (FIG. 7D) or any combination thereof. In other embodiments, any of the graphical elements 916 may be incorporated into the received image 911 itself.

In some embodiments, the display 132 may display to a user the confidence score associated with each class 542 (FIG. 5) for a received image. This data may correspond to the image 911 as a whole, segments of the image 911, or individual pixels within the image 911. The system 100 may also generate and display metrics relating to the severity of restricted blood flow of each class 542, the predicted measurement of the blood flow of each class 542, diameters of vessels at and/or around compression sites, tortuosity of various vessels, lengths of vessels or regions of stenosis, or any other suitable metrics. One or more of the metrics may be generated by the deep learning network, by image processing (pixel-by-pixel analysis, segmentation, global or local shift, warping, path solving, calibrating, etc.), other suitable technique, or combinations thereof.

At step 820, the method 800 includes recommending a stent type. Based on the graphical elements listed above and accompanying metrics output from the deep learning network as described in step 815, the deep learning network may recommend a type of stent to be used to remedy a patient's condition. In some embodiments, a user of the system 100 may input additional metrics or data in addition to the output of step 815 or the output of the deep learning network 910. The output of the step 820 can include a particular brand or type of stent, the length of the stent, and the diameter of the stent. A graphical representation 928 (FIG. 9) of the stent recommendation can be output to the display. The graphical representation 928 can be adjacent to or spaced from the image 911.

In some embodiments, a recommended stent, including, for example, any of the types of stents previously mentioned, is algorithmically predicted from a lookup table 920 of available stents. In some embodiments, the lookup table 920 may be created by a manufacture of the system 100. A user of the system 100 may be able to modify the lookup table 920. In other embodiments, the lookup table 920 may be created by experts in the field. The lookup table 920 may be a list of all available stents that have been, or may be, positioned within the iliac vein 314 (FIG. 3) or surrounding or similar vessels. The stents within the lookup table 920 may have varying lengths, foreshortening attributes, strength points, flexibility, or any other characteristic. The lookup table 920 may also be referred to as a decision tree. In some embodiments, the lookup table 920 may be implemented as a part of, or as an output of, the same deep learning network 910 previously described. The lookup table 920 may also be created based on recommendations of experts in the field. For example, if one or more experts in the field recommended a particular stent to remedy a condition with anatomical features similar to the one shown in the received image 911, the system 100 may recommend, based on an output from the deep learning network 910, the stent recommended by experts. In still other embodiments, a user may manually select a stent from the lookup table 920 based on the outputs from the deep learning network 910.

Stent selection may depend on the length, diameter, and material of the stent. At the compression site, or at or near the region of stenosis, the stent should be stiff. After the stent is positioned within the vasculature of a patient, the ends of the stent should not be close to any compression sites or regions of stenosis. The diameter of a stent may additionally determine stent selection based on the diameter of the vessel in which the stent will be positioned. Stent selection may also depend on the force required to dislodge the stent once it is positioned within a lumen. This force may be determined by the number of contact points of the vessel and the stent after it is deployed. Particularly for tortious vessels, the expanded stent may not be in physical contact with all locations of the inner lumen. In such an example, to prevent dislodging or stent migration, a longer stent may be selected to increase contact between the stent and the wall of the vessel.

At step 825, the method 800 includes generating and displaying recommended stent landing zones 926. In some embodiments, an additional mask 925 of recommended stent landing zones 926 and regions of maximum compression 927 is created algorithmically. In some embodiments, the location of the landing zones 926 is determined using the deep learning network, image processing, and/or combinations thereof. In some embodiments, the region of maximum compression 927 can be an output of the deep learning network or based on the output. These landing zones 926 may be locations within the iliac vein 314 (FIG. 3), or any other suitable vessel, where ends of a stent are to be positioned prior to engagement. The positioning of the stent may depend on several variables, such as selection of the type of stent in step 820, the mechanical properties of the stent and/or the patient anatomy, the severity, the cause, and/or the length of the blood flow restriction, and other variables. In addition, stenting across the inguinal ligament has been associated with high risk of in-stent restenosis due to poor selection of the stent type, poor placement of the stent, and high pressure exerted from the inguinal ligament. This is related to both stent placement as well as the fact that stenting across the inguinal ligament may necessitate a longer stent. The landing zones 926 may therefore account for stent foreshortening, vessel tortuosity, regions of maximum strength for the stent, use of multiple stents in long lesions, or any other suitable characteristic of the anatomy or stent. For example, if the recommend stent brand or type is stronger in the central region of the stent (as opposed to the end regions), the stent landing zones 926 can be selected such that, for the given length of stent, the stent is positioned such that the central region acts on the region of maximum compression 927. This way, the efficacy of the stent in increasing the diameter of the vessel lumen and restoring blood flow is advantageously improved, thereby improving the treatment outcome for the patient. The system 100 may generate and display to a user graphical representations of the locations of the recommended stent landing zones and/or regions of maximum compression at appropriate positions within an image (e.g., overlaid on the image).

The mask 925 may additionally depict a region of maximum strength of the stent. The system 100 may generate and display to a user a graphical representation of the locations of maximum strength of the recommended stent at appropriate positions within an image. In some embodiments, a stent may include several regions of maximum strength or may have one. For some stents, regions towards either end of the stent may be regions of decreased strength and subject to collapsing. The mask 925 may therefore direct a user to place the stent at landing zones 926 to avoid positioning regions of low strength of the stent at or near identified compression sites. The mask 925 may depict a region 927 of greatest compression. The recommended stent landing zones 926 may be placed in such a way as to position the region of maximum strength of the stent at or near this region 927 of greatest compression. For example, if a region of maximum compression 927 corresponds to the location of the inguinal ligament, the region of maximum strength would ideally be positioned within the vessel at or near the inguinal ligament.

In still other embodiments, the mask 925, as well as the recommendation of a type of stent as described in step 820, may account for the tortuosity of the iliac vein 314 and surrounding veins or regions. For example, more rigid stents must be placed with care across tortuous segments and the mask 925 may be used to identify ideal landing zones 926 to account for tortuosity. In some instances, the landing zones may be determined such that more flexible portions of the stents are positioned within the more tortuous regions of the vessel, whereas more rigid portions of the stent are positioned in more linear, less tortuous regions of the vessel. In some instances, the recommendation in step 820 may avoid rigid stents altogether for a more tortuous vessel segment, in favor of more flexible stents.

It is noted that any of the previously mentioned variables, measured or observed characteristics, and/or any of the previously mentioned outputs of the deep learning network 910 may all serve as inputs or data points for the step 825. Specifically, any of these inputs may be used to generate a mask of recommended landing zones 926 and/or one or more regions of maximum compression 927. In this way, the mask 925 may be an additional output of the deep learning network 910, may be an output of an additional deep learning network, may be an output of an additional lookup table or decision tree, or any other suitable algorithm.

At step 830, the method 800 includes highlighting anatomical landmarks within a displayed image. Certain anatomical landmarks within an anatomy of a patient may further assist a user of the system 100 to identify likely compression sites and the system 100 may accordingly highlight these anatomical landmarks. For example, notches in the pelvic bones, as shown highlighted in FIG. 7D and again in the mask 915 of FIG. 9, may assist a user of the system 100 to locate the inguinal ligaments of a patient or may assist a user to otherwise orient a view of a patient's anatomy in relation to common or distinctive structures within the anatomy. In some embodiments, as previously described, highlighting of anatomical landmarks, such as the notches of the pelvic bone, may be an additional output of the deep learning network 910 as shown. In still other embodiments, the highlighting of anatomical landmarks may be performed manually by a user of the system 100.

In some embodiments, the system 100 may additionally display to a user the locations of restrictions in blood flow in the vasculature. The system 100 may display to a user any suitable number of locations of blood flow restrictions. For example, the system 100 may display one, two, three, or more locations of restricted blood flow. These locations may be displayed to a user overlaid on a venogram image or by any other suitable method.

In some embodiments, the system 100 or a user of the system 100 may adjust the deep learning network weights at this or any other step. For example, the deep learning network weights may be dynamic and may be adjusted to suit a specific facility, imaging device, system, or patient, or may be adjusted based on any suitable environment or application. This adjustment of deep learning network weights may be referred to as a calibration.

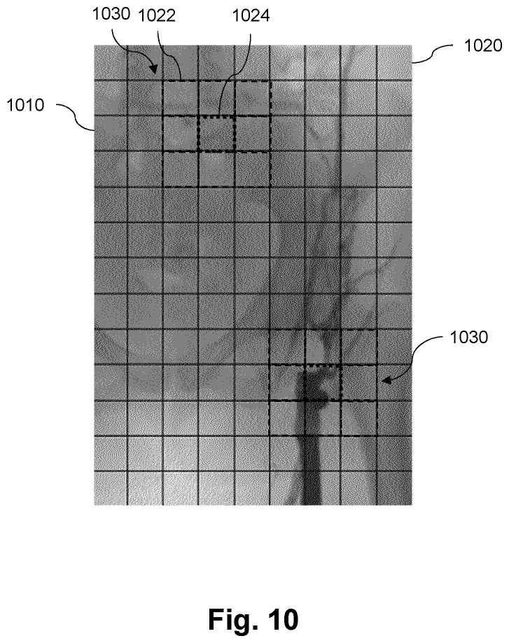

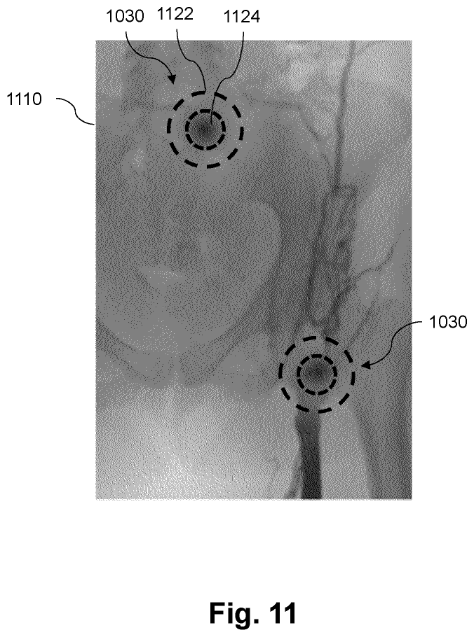

FIG. 10 is a diagrammatic view of a segmented x-ray venogram image 1010 identifying regions of interest 1030, according to aspects of the present disclosure. FIGS. 10 and 11 may represent venogram images similar to venogram images 911 previously discussed that are presented to the deep learning network 910 (FIG. 9). FIGS. 10 and 11 may represent different methods of identifying regions of interest 1030 as employed by the deep learning network 910. In some embodiments, the method described in relation to FIG. 10 may correspond to a multi-class classification network as previously described and the method of FIG. 11 may correspond to an encoder-decoder network. In other embodiments, however, any suitable type of network including multi-class classification networks, encoder-decoder networks, a patch-based classification network, a segmentation network, a regression of segmentation, or any other suitable network may analyze the images of FIG. 10 and/or FIG. 11 interchangeably. Regions of interest 1030 may include any of the previously mentioned regions such as the location of an inguinal ligament, the location of crossover of the iliac artery and the iliac vein, or other general regions of stenosis.

In an embodiment shown in FIG. 10, a received venogram image 1010 may be divided or segmented into evenly distributed and evenly sized patches 1020 such that a grid is placed over the image 1010. The patches 1020 may additionally be referred to as segments, cells, clusters, sections, or any other suitable term. Each patch 1020 may include multiple pixels of the image 1010. Each patch 1020 may be considered separately by the deep learning network, which was trained on the task of identifying any and/or all of the classes 524 (FIG. 5). The deep learning network may then classify each patch 1020. In other words, a confidence score associated with each class 542 may be assigned to each patch 1020 within the image 1010. As a non-limiting example, if the deep learning network is trained to identify three separate classes, three confidence scores would be generated by the network for each patch 1020, one associated with each of the three classes.

In some embodiments, if the deep learning network determines that the confidence score associated with a particular class 542 is exceeded within a patch 1020, the patch may be identified. In some embodiments, as shown in FIG. 10, the patch 1020 may be identified by applying a shading of a different color or opacity to the patch 1020. The color or opacity may correspond to the value of the confidence score or the level of confidence with which the network predicts the location of a compression site associated with the particular class 542. For example, a patch 1024, as illustrated in FIG. 10, may correspond to a higher level of confidence score while a patch 1022 may correspond to a lower level of confidence score but still a level which exceeds a predetermined threshold. Any suitable additional thresholds may be selected either automatically by the system 100 or user of the system 100 corresponding to various colors or opacities. In addition, any suitable number of different types of identifications may be implemented such as two different types of identifications as shown in FIG. 10 including patches 1022 and 1024 or additional numbers of different types of identifications, such as three, four, five, six, 10, 20 or more types of identifications may be used by the system 100 to identify predicted regions of compression and their severity. In addition, any suitable identification method may be used. For example, a patch may be colored or shaded in a different manner as shown. In addition, a patch may be outlined or shaded with varying patterns, gradients, as well as colors, connected to, positioned near, or otherwise associated with an arrow, flag, or other indicator, identified via any alpha-numerical text, or be otherwise identified with any suitable graphical representation. In some embodiments, the image 1010 with its various subdivided patches 1020 may not be displayed to a user. In such embodiments, patches 1020 associated with a compression site of any confidence score may not be graphically identified but otherwise identified to the system 100 for example through computer readable instructions stored on a non-transitory tangible computer readable medium, or via any other suitable method. The system 100 can use this information to determine a stent recommendation and/or stent landing zone recommendation.

FIG. 11 is a diagrammatic view of an x-ray venogram image 1110 identifying regions of interest 1030, according to aspects of the present disclosure. FIG. 11 may depict the same regions of interest 1030 as FIG. 10 but in a different manner. Contrasting with the image 1010 of FIG. 10, the received image 1110 may not be divided into patches 1020, but may be either evaluated as a whole or evaluated per pixel. For example, the deep learning network may classify each pixel of the image 1110. In other words, a confidence score associated with each class 542 (FIG. 5) may be assigned to each pixel. In such an embodiment, each pixel would have associated with it the same number of confidence scores as there are classes 542.

Similar to the identification of the patches 1020 of FIG. 10, each pixel may be identified via any suitable graphical or non-graphical representation as previously listed. For example and as shown in FIG. 11, each pixel may be shaded with predetermined color or opacity associated with a given confidence score. For example, at a point at or near a location 1124, pixels of the image 1110 may be identified as having a high likelihood of depicting a compression site. The deep learning network may analyze each pixel in relation to other surrounding pixels to identify patterns, characteristics, or features of any of the previously listed compression sites. Similarly, at or near a location 1122 within the image 1110, pixels may be identified with a different color or opacity to signify a lower confidence score or less likelihood of a predicted compression site. As stated in context of FIG. 10, any method may be used to identify pixels having any suitable confidence score including any suitable graphical representations. In an embodiment in which the image 1110 is displayed to a user of the system 100, pixels may identified with any of the previously listed graphical representations. In embodiments in which the image 1110 is not displayed to a user, pixels may be identified with any of the previously listed non-graphical representations including stored computer readable instructions. In some embodiments, the method described with reference to FIG. 11 may additionally be referred to as a segmentation, multi-segmentation, or multi-classification.

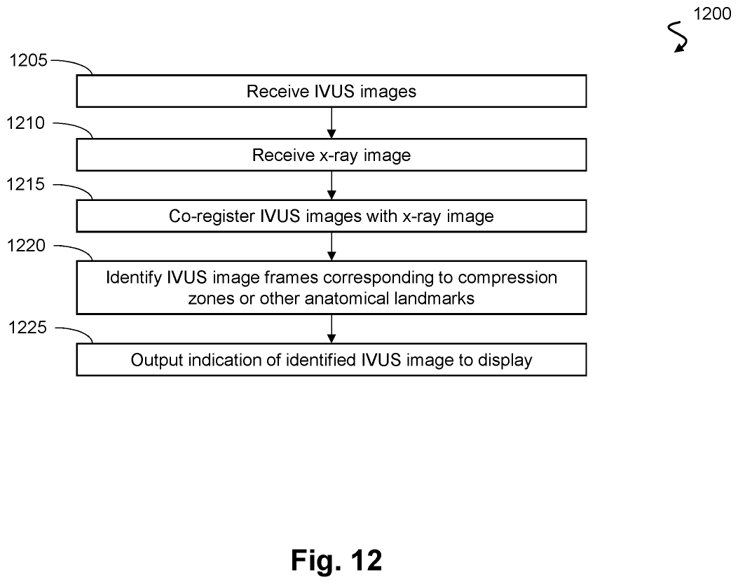

FIG. 12 is a flow diagram of a method 1200 of identifying intravascular images at locations where an intravascular imaging probe is at or near an anatomical landmark, according to aspects of the present disclosure. Examples of intravascular images and imaging probes include intravascular ultrasound (IVUS), intravascular photoacoustic (IVPA), and/or optical coherence tomography (OCT). In that regard, while IVUS is used as an example, the present disclosure contemplates any suitable type of intravascular imaging. One or more steps of the method 1200 can be performed by a processor circuit of the system 100, including, e.g., the processor 134 (FIG. 1). As illustrated, the method 1200 includes a number of enumerated steps, but embodiments of the method 1200 may include additional steps before, after, or in between the enumerated steps. In some embodiments, one or more of the enumerated steps may be omitted, performed in a different order, or performed concurrently. An enhanced method of detecting iliac vein compression involves combining and coregistering x-ray images of blood vessels with IVUS imaging. In some aspects, IVUS imaging may greatly enhance venography analysis by providing additional metrics such as vessel diameter, sizes and locations of vessel blockages, or other information. In addition, venogram images may enhance IVUS imaging by providing extravascular information such the location of an IVUS imaging probe within a vessel, the location of observed regions of stenosis within an anatomy and other information as is described with the method 1200. An example of co-registration of intravascular data and peripheral vasculature is described in U.S. Provisional Application No. 62/931,693, filed Nov. 6, 2019, and titled “CO-REGISTRATION OF INTRAVASCULAR DATA AND MULTI-SEGMENT VASCULATURE, AND ASSOCIATED DEVICES, SYSTEMS, AND METHODS”, the entirety of which is hereby incorporated by reference.

At step 1205, the method 1200 includes receiving IVUS images from an IVUS imaging probe. As previously mentioned, an ultrasound transducer array 112 positioned on an ultrasound imaging probe 110 may move through a blood vessel and emit and receive ultrasound imaging waves to create IVUS images. In some embodiments, the received IVUS images may be stored in a memory in communication with the system 100 to be recalled at a later time or may be generated and displayed and/or coregistered in real time in a point-of-care setting.

At step 1210, the method 1200 includes receiving an x-ray image. The received x-ray image may be an x-ray image, such as a venogram image. Like the received IVUS images of step 1205, the x-ray image may be generated via x-ray imaging system 151 and stored in a memory in communication with the system 100 to be recalled at a later time or may be generated and displayed and/or coregistered in real time in a point-of-care setting. In some embodiments, a patient may be examined with an IVUS imaging device 102 and with an x-ray imaging device 152 simultaneously or nearly simultaneously, at the same examination, or at different examinations.

At step 1215, the method 1200 includes co-registering the received IVUS images with the received x-ray image such that the location of an IVUS imaging probe may be measured or observed in relation to the received x-ray image. In some embodiments, co-registering the received IVUS images and received x-ray image may involve overlaying the images with one another. Co-registering images or information from the IVUS imaging system 101 and the x-ray imaging system 151 may additionally be referred to as or described as synchronizing the two modality images. As previously mentioned, aspects of the present disclosure may include features or functionalities similar to those disclosed in U.S. Pat. 6,428,930, the entirety of which is hereby incorporated by reference.

At step 1220, the method 1200 includes identifying IVUS image frames corresponding to compression zones or other anatomical landmarks. Information from the received IVUS images may be augmented with information from a previously or simultaneously created x-ray venogram image. For example, the venogram image may identify compression zones including regions at or near the inguinal ligament, the iliac artery crossover, or other regions of stenosis as well as other significant anatomical landmarks. In some embodiments, once the IVUS imaging probe reaches region of venous compression, the corresponding output ultrasound image may be identified. In some embodiments, this identification of an output IVUS image may trigger additional tools or measurement methods to acquire various metrics of the vessel. For example, the IVUS imaging probe may calculate the vessel diameter, vessel area, lumen diameter, lumen area, blood flow within the vessel, the size and location of vessel blockages, or any other metrics using any suitable measurements tools. The additional information obtained by the IVUS imaging probe coregistered with the input venogram may provide additional inputs to the deep learning network to help it more accurately identify regions of venous compression. In some embodiments, the system 100 may use image processing techniques such as quantitative coronary angiography (QCA) or other processing techniques to calculate any of the previously mentioned metrics such as vessel diameter, lumen diameter, vessel length, compression length, or other dimensions.

At step 1225, the method 1200 includes outputting an indication of an identified IVUS image to the display 132. In some embodiments, the system 100 may identify any received IVUS images that are at or near a compression site or other anatomical landmark via a graphical representation. The graphical representation used to identify the IVUS image may be of any suitable type including any previously listed graphical representation. In addition, the graphical representation may display to a user one or more metrics associated with the IVUS image or the coregistered venogram image. For example, the type of graphical representation used may correspond to the distance of the IVUS probe from a region of compression. For example, the graphical representation may vary in color, size, gradient, opacity, pattern, or by any other characteristic, as the IVUS probe approaches or moves away from a region of compression. In some embodiments, the graphical representation may additionally denote the type of region of compression the IVUS imaging probe may be at, near, and/or approaching. The graphical representation may additionally convey to the user any of the previously discussed metrics of the imaged vessel including but not limited to the diameter of the vessel, predicted blood flow, the severity of compression of the region, among others.

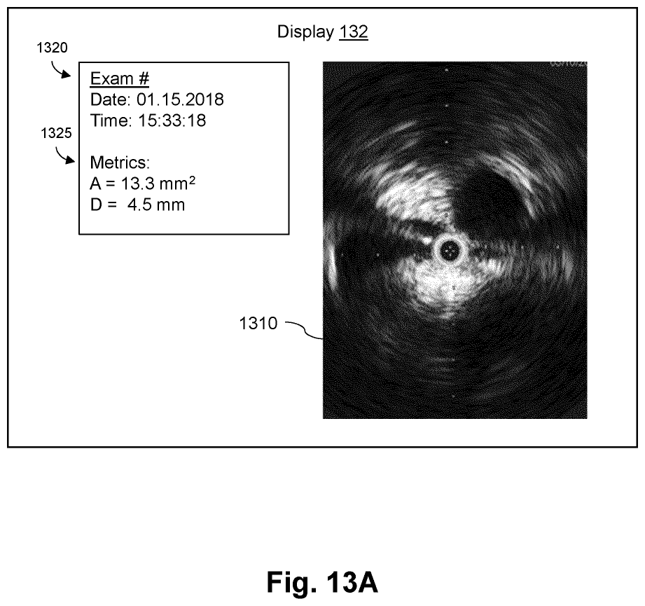

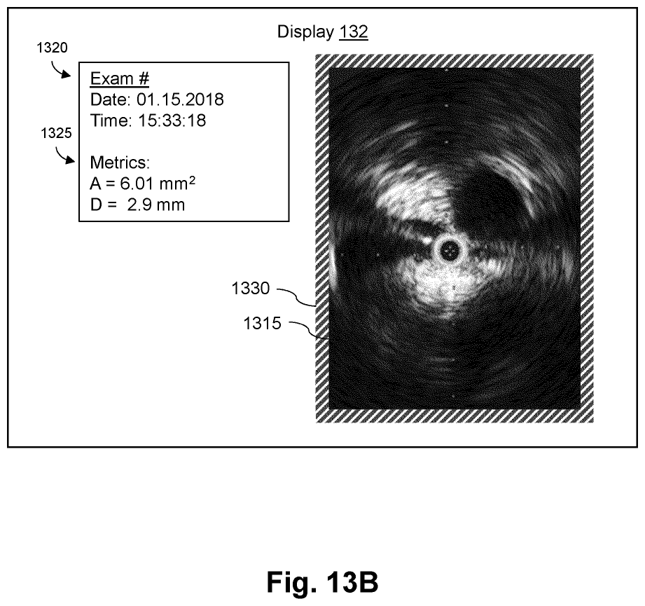

FIG. 13A is a diagrammatic view of a graphical user interface displaying an IVUS image at a location where the IVUS imaging probe is not near an anatomical landmark, according to aspects of the present disclosure. FIGS. 13A and 13B may provide an example representation of a graphical user interface as seen by a user of the system 100. As described by the method 1200 with reference to FIG. 12, individual IVUS image frames may be identified or not identified based on their proximity to regions of compression or other anatomical landmarks among other characteristics. At a location that is not near a region of compression, the display 132 may depict to a user an IVUS image frame 1310. The IVUS image frame 1310 may be received, processed, and displayed by the control system 130.

In some embodiments, whether an IVUS imaging frame is to be identified as near a region of compression or other anatomical landmark may be determined by a threshold distance. For example, the manufacturer of the system 100 may select a threshold distance. When the IVUS imaging probe is positioned within this predetermined threshold distance to a region of compression or other anatomical landmark, the system 100 may identify the associated IVUS imaging frame(s) as such. Alternatively, this threshold may be determined by the deep learning network, experts in the field, or a user of the system 100.

In addition to identifying IVUS imaging frames in close proximity to regions of compression or anatomical landmarks, the system 100 may additionally use one or more outputs of the deep learning network previously described to automatically highlight, annotate, or select IVUS image frames and measurements.

In some embodiments, other general information 1320 relating to the exam or any other suitable information as well as metrics 1325 related to the imaged vessel may be displayed to a user. The display 132 may display this information 1320 and/or metrics 1325 adjacent to, to the side of, above, below, or overlaid on the IVUS image 1310. General information 1320 relating to the examination may include such metrics as the exam number, indicating how many examinations have been performed on the anatomy of a given patient, the date and time of the exam, as well as any other suitable information. For example, other information may include data to patient history, past or current diagnoses or conditions, past or current vital signs of a patient being examined, or any other useful information. In addition, the metrics 1325 may include any suitable metrics previously listed, including blood flow, cross section area of the vessel or lumen, diameter of the vessel or lumen, or any other measurable metrics. In some embodiments, the IVUS imaging probe may additionally be used to examine or survey vessel damage or trauma at various locations within a patient's vasculature and may display additional general information or metrics associated with any measured damage.

FIG. 13B is a diagrammatic view of a graphical user interface displaying an IVUS image 1315 at a location where the IVUS imaging probe is near an anatomical landmark, according to aspects of the present disclosure. FIG. 13B may be substantially similar to FIG. 13A in that it displays a graphical user interface displaying an IVUS image 1315. However, the primary difference between FIG. 13A and FIG. 13B may be an additional graphical representation 1330. The graphical representation 1330 may indicate to a user that the IVUS imaging probe is at or near a region of compression or anatomical landmark. As mentioned with regards to the step 1225 of the method 1220, the graphical representation 1330 may be any suitable graphical representation including all of the previously listed examples. In addition, the graphical representation 1330 may convey to a user any other metrics or information relating to the position of the IVUS imaging probe in relation to any anatomical features within the anatomy, dimensions or conditions of the imaged vessel, or any other previously mentioned or suitable characteristic, information, metric, or feature. In that regard, metrics associated with the vessel or vessel lumen (e.g., area and/or diameter) can be automatically provided to the user in response to identifying one or more IVUS images that are near a region of blood flow restriction.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an external imaging device, wherein the processor circuit is configured to:
receive, from the external imaging device, an image comprising a blood vessel within a patient;
determine, using the image, a first location of the blood vessel with a restriction in blood flow caused by compression of the blood vessel by an anatomical structure within the patient and different than the blood vessel;
generate a first graphical representation associated with the restriction;
output, to a display in communication with the processor circuit, a screen display comprising:
the image; and
the first graphical representation at the first location of the blood vessel in the image.

2. The system of claim 1, wherein the external imaging device comprises an x-ray imaging device, and wherein the image comprises an x-ray image.

3. The system of claim 1, wherein the processor circuit is configured to determine the first location of the blood vessel with the restriction using a convolutional neural network.

4. The system of claim 3, wherein the convolutional neural network is trained using a plurality of images with identified restrictions in blood flow caused by the compression of further blood vessels by further anatomical structures.

5. The system of claim 3, wherein the processor circuit is configured to classify the first location of the blood vessel with the restriction as a first type of restriction or a second type of restriction.

6. The system of claim 5, wherein the first type of restriction comprises a location of a ligament and the second type of restriction comprises a crossover of the blood vessel and a further blood vessel.

7. The system of claim 3, wherein the processor circuit is configured to segment anatomy within the image.

8. The system of claim 3, wherein the processor circuit is configured to:
divide the image into a plurality of patches, wherein each patch of the plurality of patches comprises a plurality of pixels of the image; and
determine a patch as the first location of the blood vessel with the restriction.

9. The system of claim 1,
wherein the image comprises a first image,
wherein the processor circuit is configured to receive a second image comprising at least one of the blood vessel or the anatomical structure, and
wherein the processor circuit is configured to determine the first location of the blood vessel with the restriction using the first image and second image.

10. The system of claim 9,
wherein the first image comprises a first x-ray image obtained with contrast within the blood vessel, and
wherein the second image comprises a second x-ray image obtained without contrast within the blood vessel.

11. The system of claim 9,
wherein the first image comprises an x-ray image,
wherein the second image comprises an intravascular ultrasound (IVUS) image,
wherein the processor circuit is configured for communication with an IVUS catheter,
wherein the processor circuit is configured to receive the IVUS image from the IVUS catheter.

12. The system of claim 1, wherein the first graphical representation comprises a color-coded map corresponding to a severity of the restriction in the blood flow.

13. The system of claim 1, wherein the processor circuit is configured to:
determine a stent recommendation to treat the restriction based on at least one of the image or the first location of the blood vessel with the restriction; and
output the stent recommendation to the display.

14. The system of claim 13, wherein the processor circuit is configured to:
determine a stent landing zone at a second location of the blood vessel based on at least one of the stent recommendation, the image, or the first location of the blood vessel with the restriction;
generate a second graphical representation of the stent landing zone; and
output the second graphical representation at the second location of the blood vessel in the image.

15. The system of claim 14, wherein the processor circuit is configured to:
determine a stent strength position at a third location of the blood vessel based on at least one of the stent landing zone, the stent recommendation, the image, or the first location of the blood vessel with the restriction;
generate a third graphical representation of the stent strength position; and
output the third graphical representation at the third location of the blood vessel in the image.

16. The system of claim 1,
wherein the processor circuit is configured for communication with an intravascular ultrasound (IVUS) catheter,
wherein the processor circuit is configured to:
receive a plurality of IVUS images along a length of the blood vessel from the IVUS catheter,
co-register the plurality of IVUS images with the image;
identify an IVUS image of the plurality of IVUS image corresponding to the first location of the blood vessel with a restriction; and
output the IVUS image to the display.

17. A blood vessel compression identification system, comprising:
an x-ray imaging device configured to obtain an x-ray image comprising a vein within a patient; and
a processor circuit in communication with the x-ray imaging device, wherein the processor circuit is configured to:
receive the x-ray image from the x-ray imaging device;
determine, using a deep learning algorithm, a first location of the vein with a restriction in blood flow caused by compression of the vein by an anatomical structure within the patient and different than the vein, wherein the anatomical structure comprises an artery or a ligament;
determine a stent recommendation to treat the restriction based on at least one of the x-ray image or the first location of the vein;
determine a stent landing zone at a second location of the vein based on at least one of the stent recommendation, the x-ray image, or the first location of the vein;
output, to a display in communication with the processor circuit, a screen display comprising:
the x-ray image;
a first graphical representation of the stent recommendation; and
a second graphical representation of the stent landing zone overlaid on the x-ray image at the second location of the vein.
